(19) United States
(12) Patent Application Publication
(10) Pub. No.: US 2022/0039679 A1
(43) Pub. Date: Feb. 10, 2022

(54) SYSTEM AND METHOD FOR REMOTE MONITORING OF BIOMEDICAL PARAMETERS

(71) Applicant: CONTINUSE BIOMETRICS LTD., Tel Aviv (IL)

(72) Inventors: Ran CALIFA, Givataym (IL); Hadar GENISH, Petah-Tikva (IL); Matan BENYAMIN, Ramat Gan (IL); Eran HOCHSTADTER, Kibbutz Haogen (IL); Javier GARCIA, Valencia (ES); Yevgeny BEIDERMAN, Tel Aviv (IL); Michael SHERGEI, Petah Tikva (IL); Zeev ZALEVSKY, Rosh HaAyin (IL)

(21) Appl. No.: 17/297,673

(22) PCT Filed: Nov. 27, 2019
(86) PCT No.: PCT/IL2019/051297

§ 371 Date: May 27, 2021

Related U.S. Application Data
(60) Provisional application No. 62/771,692, filed on Nov. 27, 2018.

(51) Int. Cl. A61B 5/024; H04N 5/235; H04N 5/225; H04N 5/247; G06T 7/70; G06T 7/20; G06K 9/20; G06K 9/00; G06T 7/00; A61B 5/021; A61B 5/0205; A61B 5/08; A61B 5/00

(52) U.S. Cl. CPC A61B 5/02427; G06T 2207/30076; H04N 5/2256; H04N 5/247; G06T 7/70; G06T 7/20; G06K 9/2027; G06K 9/00832; G06T 7/0012; A61B 5/02108; A61B 5/0205; A61B 5/0816; A61B 5/02438; A61B 5/6893; A61B 5/6801; G06T 2207/10016; G06T 2207/30201; G06T 2207/30268; G06T 2207/10144; G06T 2207/10152; H04N 5/2352

(57) ABSTRACT

A system for monitoring one or more biological parameters of an individual, the system comprising: an illumination unit comprising at least one coherent light source configured for providing at least one coherent optical illumination beam and for directing said beam onto a selected inspection region on body of said individual; a light collection unit comprising at least a first imaging unit, said first imaging unit comprising a lens unit and a detector array and configured for collecting light returning from said inspection region to thereby generate at least one sequences of image data pieces associated with said inspection region, at a selected sampling rate; a control unit comprising at least one processor, the control unit is configured for receiving input data comprising said at least one sequence of image data piece and for processing the image data pieces for generating data indicative of one or more parameters of the individual. The processing comprises determining contrast variations in image data comprising image data indicative of one or more speckle patterns formed in light returning from illumination spots in said inspection region.

### TECHNOLOGICAL FIELD

The present invention relates to systems and methods for remote monitoring of biomechanical and biomedical parameters of an individual. The technique specifically relates to monitoring of health-related parameters in noisy environment such as moving vehicles.

### BACKGROUND

In previous years, various techniques have been developed for remote sensing and monitoring of biological and biomechanical parameters. Such techniques allow continuous measurements of subject's parameters (e.g. medical and/or biological parameters) and with minimum disturbance to the subject.

Remote photoplethysmographic (PPG) techniques (rPPG) gained interest in recent years, providing remote blood pulsation monitoring that can later be used to extract information about patient health, mood, and more. Most of the PPG and rPPG techniques include measurements of color variations associated with changes in blood volume and oxygenation in the subject's blood at a selected specific anatomical site being measured.

Additional remote sensing techniques are also known, enabling remote monitoring of subjects' biomedical parameters from a distance. Some of such techniques utilize analysis of speckles formed in coherent illumination reflected/scattered from the subject's body. Dynamic speckles are a physical mechanism in which motions of scattering particles in the medium can be evaluated by analyzing the time dependency of speckle patterns in light reflected from, or transmitted through, the medium. Dynamic speckles may be used and analyzed by directly measuring the time evolution of the speckle correlation as well as by measuring the time evolution of statistical properties such as contrast of the speckle pattern.

Generally, Speckles are a form of self-interference phenomenon occurring when a coherent field is scattered from rough objects. When coherent electromagnetic radiation, e.g. laser beam, is backscattered from an optically rough medium, such as biological tissues, a speckle pattern may appear in the backscattered light. When the tissue changes, in our case due to blood pulsation, the speckle pattern will change accordingly. This time evolution of the pattern is often known as dynamic speckle. Tracking the dynamics of the speckle pattern can reveal useful information about the behavior of the scattering medium.

### General Description

As indicated above, optical remote monitoring of biological parameters may provide robust and efficient technique for monitoring biomedical parameters of subjects/patients within or outside of medical facilities. Although the above mentioned dynamic speckles monitoring techniques may be used in various conditions, these techniques generally fail in high noise environments such as within moving vehicles or as part of wearable device, where vibrations cause movement of both the subject and the monitoring system introducing high noise to the collected data.

The present invention utilizes dynamic speckles monitoring while being configured for operation in high noise (generally vibrations) environments. To this end the present technique utilizes laser speckle contrast analysis (LASCA) technique for monitoring blood flow in a subject's body, thereby enabling to determine biological parameters such as heart rate, heart rate variability (HRV), blood pressure (BP) and pulse wave velocity (PWV) of a subject in different environments. In some embodiments, the present technique utilizes monitoring of one or more different regions on body of an individual, enabling detection of pulse transition time and pulse wave velocity by determining distance measure between two or more different inspection positions. Generally, the present technique may also utilize combined monitoring using contrast analysis (LASCA) at one or more inspection regions as described herein and speckle correlation analysis at one or more additional inspection regions.

More specifically, the present technique is based on directing coherent illumination of a selected wavelength range onto one or more inspection spots of the subjects' body, using a first imaging unit and collecting one or more sequences of image data pieces, associated with images of one or more selected inspections spots, and processing the collected image data for determining variations in contrast of speckle patterns in the image data pieces. The first imaging unit may preferably be operated with sampling rate sufficient for measuring inter-beat intervals (IBI) allowing monitoring of heart rate while providing sufficient measurement resolution for determining heart rate variability of the subject. For Example, the first imaging unit may operate at sampling rate of 100-300 frames per second.

Accordingly, the present invention provides a system for remote monitoring parameters of an individual, the system generally comprises an illumination unit configured for directing coherent illumination onto a selected inspection region of the subject's bode, thereby forming one or more illumination spots on the subject. The system further comprises a collection unit comprising at least a first imaging unit configured for collecting one or more sequences of image data pieces associated with images of the illumination spot and providing the sequence of image data pieces for processing. A control unit, generally comprising at least one processor and storage utility, is configured for receiving input data in the form of one or more sequences of image data pieces collected by the first imaging unit, and for processing the input data to determine one or more selected parameters of the subject. The present technique may typically utilize illumination of non-visible wavelength such as near infrared (NIR) for eye safety and to avoid generating distractions to the subject.

Generally, speckle patterns are recorded by collecting image data pieces with certain finite exposure time. During the exposure time, changes in the inspection region collected in the image data pieces (by scattering light therefrom) result in blurring of the collected speckle pattern. Further, the level of blurring is associated with rate of changes on the inspection region. Thus, determining contrast measure for each image data piece and collecting the contrast measures from a sequences of image data pieces enables determining a time-contrast variation function indicative of varying rate of changes in the inspection region. When the inspection region ins a portion of human skin, such changes are indicative of capillary blood perfusion and provide data on cardiac activity (e.g. heart rate) of the subject.

The use of speckle contrast analysis according to the present technique enables monitoring of subjects' parameters in moving vehicles and/or using wearable devices while the subject moves. In such applications, the present technique provides various advantages over the conventional techniques for monitoring physiological parameters (e.g. PPG). For example, PPG techniques provide better performance when monitoring body portions characterized with high blood volume, and the performance is reduced when the blood volume reduces (e.g. in cold conditions). Differently, the present invention is based on monitoring of blood flow and movement and may thus provide efficient monitoring of any body portion and is not affected by temperature. Moreover, the present technique utilizes the selected, and relatively short, exposure window providing robustness to vibrations and mechanical noise.

To overcome environmental noise, generally associated with vibrations such as in a moving vehicle, the present technique generally utilizes short exposure time for imaging collection by the first imaging unit. Indeed, capturing images using short exposure time enables filtering out relatively slow movements between the system and the inspection region. Further, the inventors have found that capillary blood flow provides high speed variations as compared to mechanical movement of the subject (and the system) due to mechanical vibrations, e.g. such as associated with moving vehicles. Thus, the use of short exposure time reduces both noise associated with mechanical vibrations in the environment as well as those associated with movement of the subject, while enabling to determine data on capillary blood flow by contrast speckle analysis. Further, the system may also be used in wearable devised, allowing collection of biological parameters of the subject (wearing the device) while generally not interfering with any other activity the subject is performing.

In some configurations, the first imaging unit may be configured from two or more imaging sub-units capable of being separately operated with different imaging parameters. The different imaging sub-units are operating in different exposure times and/or frame rates to allow collected of spackle patterns with different characteristics and thus allow overcoming of increase vibrational noise and detection of the desired signal.

Further, the present technique may utilize processing of selected regions of the speckle pattern separately. More specifically, the processor unit may operate for determining, within received image data pieces, speckle regions associated with the illumination spot and having speckle patterns. The processor unit may divide the speckle region to two or more sub-regions and determine separate contrast measure for the different sub-regions. This provides a plurality of two or more time-contrast variation functions corresponding with the different sub-regions. Analysis of the different time-contrast variation functions and determining weighted average thereof may provide for determining the one or more selected parameters with improved signal to noise ratio. Further, due to mechanical vibrations in the environment, location of the illumination spot may change with respect to the inspection region. The present technique may utilize tracking of the relative locations of the illumination spot and inspection region to thereby identify similar sub-regions being illumination and maintain the sub-region time-contrast variation function for selected spots on the inspection region, even when the illumination spot shifts. The tracking may generally be based on image data associated with video data of the surrounding of the subject.

To this end the system of the present invention may utilize an additional, second, imaging unit. The second imaging unit is preferably configured with relatively wide field of view, enabling collecting of image data of the subject, where the inspection region and illumination spot occupy a portion of the field of view. The second imaging unit may be configured for operating with sampling rate that is slower with respect to that of the first imaging unit. Image data collected by the second imaging unit is transmitted to the control unit for processing, e.g. using a location tracking module, and for identifying relative location between the illumination spot and the inspection region. Tracking of the relative location may be used for determining drift of the illumination spot with respect to the inspection region. Such drift may be associated with shift of sub-regions that can be corrected by following selected sub-regions on basis of the inspection region thereby compensating for shifts of the illumination spot. In some other cases such drift may be fixed by physical alignment of the system.

In some other configurations, the illumination unit may comprise, in additional to the coherent illumination light source, a wide field illumination source (e.g. LED light source) and configured for providing intervals of coherent illumination pulses separated between them by pulses of field illumination. The first imaging unit may thus collect sequence of interleaving image data pieces and split the collected frames based on illumination conditions, where frames illuminated by coherent illumination are directed for speckle pattern analysis and frames illuminated by field illumination are directed to a video channel. The video channel is transmitted to the control unit for processing, e.g. using a location tracking module, and for identifying relative location between the illumination spots and the inspection region.

Additionally, the present technique may also utilize one or more additional monitoring units including for example rPPG monitoring, spatial dynamic speckle monitoring etc. Further, the collection unit may comprise one or more additional imaging units, suitable for collecting image data of the inspection region, while utilizing additional techniques for monitoring parameters of the subject. For Example, the collection unit may comprise a defocused imaging unit configured for collecting image data while being out-of-focus with respect to the inspection region. Accordingly, the defocused imaging unit is configured for providing at least one second sequence of image data piece to the control unit for additional processing. The processing of the second sequence of image data piece comprises determining spatial correlations between image data piece and accordingly determining corresponding time-correlation function indicative of vibrations of the inspection region providing speckle correlation analysis. Such time-correlation function may be affected by noise associated with mechanical vibrations of the subject (e.g. in a moving vehicle), however it may be used to provide reference data on the one or more parameters to be determined.

Thus, according to a broad aspect, the present invention provides a system for monitoring one or more biological parameters of an individual, the system comprising:

An illumination unit (e.g. light source unit) comprising at least one coherent light source configured for providing at least one coherent optical illumination beam and for directing said beam onto a selected inspection region on body of said individual;

a light collection unit comprising at least a first imaging unit, said first imaging unit comprising a lens unit and a detector array and configured for collecting light returning from said inspection region to thereby generate at least one sequences of image data pieces associated with said inspection region, at a selected sampling rate;

a control unit comprising at least one processor, the control unit is configured for receiving input data comprising said at least one sequence of image data piece and for processing the image data pieces for generating data indicative of one or more parameters of the individual wherein said processing comprises determining contrast variations in image data comprising image data indicative of one or more speckle patterns formed in light returning from illumination spots in said inspection region. In some configurations the determined parameters may comprise at least one of heart rate, heart rate variability, respiratory rate, blood pressure (BP) and pulse wave velocity (PWV).

According to some embodiments, said processing comprising detecting, within image data pieces of said sequence of image data pieces, region associated with image of illumination spot, said region comprises speckle pattern formed by interference of light returning from the selection illumination spot on the individual's body, and for determining, for at least a portion of said speckle pattern a contrast measure indicative of contrast of speckles in said speckle pattern, thereby generating contrast varying function associated with variation of said contrast measure through time of monitoring, said contrast varying function being indicative of said one or more biological parameters of the individual.

The first imaging unit may be configured for operating at short exposure times, thereby filtering out noise associated with vibrations of said individual. The exposure time may not exceed 1000 μs; or may not exceed 500 μs; or may not exceed 100 μs; or may not exceed 50 μs.

According to some embodiments, the first imaging unit comprises two or more separately operable imaging sub-unit operating with two or more different exposure time profiles.

According to some embodiments, the first imaging unit has a field of view directed at said inspection region, the system further comprises at least a second imaging unit comprising at least one lens unit and detector array, said second imaging unit being configured for collecting illumination from a field of view being larger with respect to said first imaging unit.

The control unit may comprise a location tracking module configured and operable for receiving image data from said second imaging unit and for tracking location of illumination spot on said inspection region to thereby identify shifts in said illumination spot. The location tracking module is configured for tagging one or more portions of the inspection region

According to some embodiments, the light source unit may comprise one or more light sources providing coherent illumination with two or more different wavelengths within a selected wavelength range, said control unit is configured for receiving and processing image data collected in said two or more different wavelength and for generating two or more corresponding contrast varying functions associated with the two or more different wavelengths. The control unit may further determine data one said one or more parameters in accordance with said two or more contrast varying functions.

According to some embodiments, the system may further comprise at least one additional imaging unit configured for collecting defocused image data from the inspection region, and for processing said defocused image data for obtaining additional data indicative of said one or more biological parameters of the individual.

According to some embodiments, the system may further comprise at least one additional imaging unit configured for Remote photoplethysmographic monitoring for obtaining additional data indicative of said one or more biological parameters of the individual.

According to some embodiments, the illumination unit may further comprise at least one field illumination light source configured for providing wide field illumination of region for inspection; said illumination unit is adapted for operating in sequential pulses of said coherent light source and said field illumination light source.

The first imaging unit may be adapted for collecting image data in synchronization with repeating pulses of said illumination unit thereby collecting image data frames associated with coherent illumination for at least one sequence of image data piece, and image data frames associated with field illumination for generating video channel enabling detection of object or face detection in the field of view. The exposure time of the first imaging unit may be greater than 500 μs, pulse length of said coherent illumination does not exceed 500 μs.

According to some embodiments, the at least one coherent light source may comprise an array of coherent light sources adapted for providing a pattern of a plurality of illumination spots within a selected field of view. The control unit may be adapted for selecting one or more illumination spots within said at least one sequence of image data pieces and processing pattern of said one or more illumination spots for determining said one or more parameters.

According to some embodiments, the first imaging unit may be adapted for wide field imaging, thereby enabling detection of one or more illumination spots from image data collected thereby.

The system of the present invention may be configured to be mounted within a vehicle for monitoring one or more individuals within the vehicle while moving. Alternatively or additionally, the system may be configured for use in wearable device for monitoring one or more parameters of an individual. For example, when mounted in a vehicle, the system may be adapted for determining presence of one or more passengers within the vehicle based on existence of one or more biomedical parameters detected within the vehicle.

According to another broad aspect, the present invention provides method for use in monitoring one or more parameters of an individual, the method comprising: illuminating at least one body portion of the individual with coherent illumination and collecting at least one sequence of image data pieces from said at least one body portion, processing the at least one sequence of image data pieces for determining at least one contrast parameter for each image data piece, thereby determining at least one time-contrast function being indicative of one or more parameters of the individual.

Said collecting at least one sequence of image data pieces may comprise operating an imaging unit at high exposure time, not exceeding 1000 μs; or not exceeding 500 μs; or not exceeding 100 μs; or not exceeding 50 μs.

The method may further comprise monitoring movement of the individual body for following a region associated with illumination spot on at least one body portion of the individual for tracking location of the illumination spot.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 schematically illustrates a system for monitoring biological parameters according to some embodiments of the invention;

FIG. 2 schematically illustrates a control unit suitable for use in a system according to some embodiments of the present invention;

FIG. 3 exemplifies a method of monitoring biomedical parameters of an individual according to some embodiments of the present invention;

FIG. 4 exemplify configuration of experimental validation for the technique of the present invention;

FIG. 5 shows experimental results for heart rate data measured by ECG, the present technique, rPPGr and rPPGt techniques;

FIGS. 6A to 6C show comparison between heart rate measured by the present technique (FIG. 6A), rPPGr (FIG. 6B), and rPPGt (FIG. 6C) to those measured by ECG;

FIGS. 7A to 7C show correlation inter beat intervals (IBI) as determined by the present technique (FIG. 7A) and by rPPGr (FIG. 7B) and rPPGt (FIG. 7C) with respect to ECG;

FIGS. 8A to 8C show Bland-Altman plots for measurements by the present technique (FIG. 8A), rPPGr (FIG. 8B) and rPPGt (FIG. 8C); and

FIG. 9 shows experimental results for heart rate data measured by the present technique, rPPGr and rPPGt techniques on a moving finger.

### DETAILED DESCRIPTION OF EMBODIMENTS

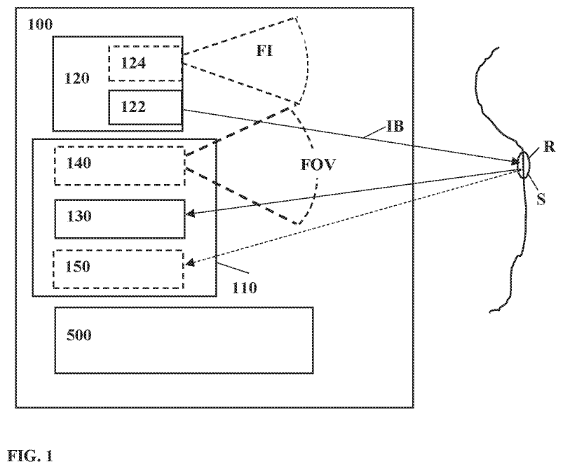

As indicated above, the present invention provides a technique and a system for monitoring biological parameters of a subject, suitable for operation in mechanically noisy environment. Reference is made to FIG. 1 schematically illustrating a system 100 according to some embodiments of the invention. System 100 includes at least one illumination unit 120, a light collection unit 110 and control unit 500. The illumination unit 120 includes at least one coherent light source 122 configured for providing at least one coherent optical illumination and directing the illumination beam IB toward a selected inspection region R on body of the subject to thereby form an illumination spot S on the inspection region R. The inspection region R is preferably a region of exposed skin. The illumination unit may also include a field illumination light source 124 configured for selectively provide field illumination FI directed at the inspection region R and its surrounding. The light collection unit 110 includes one or more imaging unit directed at the inspection region and possibly its vicinity and generate one or more sequences of image data piece based on collected light. The illumination unit 120 may generally utilize coherent light source 122, e.g. laser light source, configured for providing optical illumination of a selected wavelength range. In some configurations the laser light source 122 may be configured to provide CW illumination, or illumination with pulsed profile, at wavelength range selected to penetrate at least a few micrometers through human skin to thereby provide image data indicative of capillary blood flow. In some configurations the laser light source 122 may be configured to provide optical illumination in non-visible wavelength range, thereby reducing any danger of eye damage. More specifically, the use of near Infrared illumination, e.g. wavelength range of 800-1550 nm may be preferred providing certain penetration through the skin and being non-visible illumination. The laser light source may be configured to provide linearly polarized illumination having predetermined polarization orientation. Generally, in some configurations, the illumination unit 120 may utilizes illumination using two or more wavelengths within a selected wavelength range. This allows the present technique to monitor contrast variations in two or more different wavelengths and/or combined contrast variations for the different wavelength.

As indicated above, the illumination unit 120 may also include a field illumination light source 124. The field illumination light source 124 may for example be a LED light source, configured for illuminating a wide region by field illumination FI, generally at near Infrared illumination or visible illumination. The illumination unit 120 may operate the laser light source 122 and the field illumination light source 124 in sequence of interleaved pulses to allow collection of image data pieces multiplexing image frames associated with speckle patterns formed by the coherent illumination and image frames associated with field illumination providing general image of the region, e.g. forming a video channel.

The laser light source 122 may be configured to provide array of coherent illumination beams to thereby generate illumination spots on a plurality of inspection regions R. for example, the laser light source 122 may be formed by a laser array and/or a diffractive element configured for splitting the coherent illumination to form a plurality of illumination spots. Generally, the use of a plurality of illumination spots enables determining time-contrast function as described further below for a number of two or more illumination spots, thereby enabling determining additional parameters such as blood pressure (BP) and pulse wave velocity (PWV) by correlating relative contrast variations in the two or more illumination spots.

The collection unit includes at least a first imaging unit 130 formed of at least a detector array and imaging lens arrangement. The first imaging unit 130 is configured for collecting images of the inspection region R, to generate a sequence of image data pieces at a first sampling rate and to transmit the so-generated image data pieces to the control unit. The first imaging unit 130 is preferably configured for collecting a sequence of images at selected sampling rate, where each image is collected within a selected exposure time, such that the sampling rate is sufficient for monitoring blood perfusion and heart rate of the subject and the exposure time is sufficiently short for filtering out mechanical vibrations from the environment. For example, the first imaging unit 130 may operate at sampling rate of 30-300 frames per second. Further, the first imaging unit 130 may operate to capture images with exposure time of 500 μs or less, preferably 100 μs or less, and more preferably 50 μs or less. In some configurations, the first imaging unit 130 may be configured for operating with exposure rate longer than 500 μs, while the laser source 122 is configured to provide pulsed illumination having illumination period of 500 μs or less, thus limiting the effective exposure time by limiting illumination of the inspection region R. The first imaging unit 130 may also include a polarizer mounted to be orthogonal to polarization of illumination provided by the light source, to allow collection of light associated with reflection from internal layers of the inspection region tissue, i.e. a few micrometers to a few millimeters deep. As indicated above, the first imaging unit 130 may be formed by two or more imaging sub-units operable with different imaging parameters. For example, the different sub-units may be operable with different exposure time and/or frame rates for provide additional filtering and flexibility and enable detection of the desired biological signal under high noise interference.

The first imaging unit 130 may be configured with field of view directed at the inspection region R. More specifically, the first imaging unit 130 is preferably configured with narrow field of view such that the illumination spot S on the inspection region R takes 50% or more of the field of view. This allows collection of light returning from the inspection region R with high resolution to speckles in the collected light. Alternatively, the first imaging unit 130 may be configured with field of view covering a region in from of the light collection unit 110, e.g. capable of collecting image data of the subject body when positioned in a vehicle seat. The first imaging unit 130 may thus collecting image data with field of view sufficient for providing a complete image of the subject and surrounding thereof (e.g. including the subject's body, head etc.). Thus, the collected field of view may include within the image frame a plurality of illumination spots (e.g. where the laser light source 122 is configured to provide a plurality of illumination spots). Further, in some embodiments, the first imaging unit 130 may be configured for large area coverage having wide field of view, e.g. using fisheye lens, lenses and/or optical funnel. Such configuration combined with illumination unit 120 configured to provide a plurality of coherent illumination spot (using laser array of laser with diffractive element in light source 122) may be used for monitoring one or more subjects in selected volume/region. The technique may select one or more illumination spots for processing based on face tracking in video image data as described in more detail below.

As indicated above, the illumination unit 120 may utilize illumination using two or more different wavelength within a selected wavelength range. In such configurations, the first imaging unit 130 may be configured for polychromatic imaging or configured as two or more imaging sub-units, allowing separate imaging of the two or more wavelengths. Generally, the first imaging unit 130 may utilizes a wavelength selective filter, selected to allow transmission of light associated with the coherent illumination of laser source 122, i.e. with bandwidth of 10-20 nm around the selected wavelength. For example, when using coherent illumination at 850 nm, the first imaging unit 130 may include a chromatic filter transmitting transmitting at 850 nm±5 nm.

The light collection unit 110 may also include one or more additional imaging units such as second imaging unit 140, as well as third or fourth imaging units (illustrated at 150). The second imaging unit 140 is configured with relatively wide field of view and directed for imaging relatively large area around the inspection region R, e.g. including the subject's body, head etc. The second imaging unit 140 also transmitted at least one sequence of image data piece, typically collected at frame rate lower than that of the first imaging unit, to the control unit 500 for processing by tracking location of the illumination spot S with respect to the inspection region R as described in more details further below.

Additionally, third and/or fourth imaging units, marked as 150, may be used to provide one or more additional modes of monitoring parameters of the subject. The third imaging unit is configured for collecting defocused images of the inspection region, to thereby allow monitoring of vibrations of the inspection region by determining correlation between speckle patterns. The fourth imaging unit may be operable at a different wavelength range and configured for use in rPPG monitoring. To this end the illumination unit 120 may include an additional light source (e.g. LED light source) configures for use in rPPG monitoring.

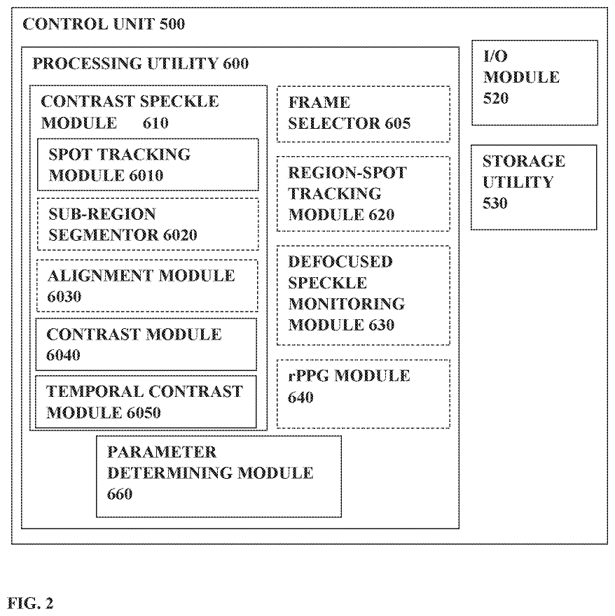

The control unit 500 is configured for operating the system 100 by providing operational commands to the illumination unit 120 and collection unit 110, and for receiving input data including one or more sequences of image data pieces for processing and determining one or more parameters of the subject. Generally, the control unit may be configured as a computer unit or system including one or more processors, memory utility and input/output modules. Reference is made to FIG. 2 schematically illustrating a control unit 500 according to some embodiments of the invention and some of its software or hardware modules.

Generally, the control unit 500 may include separate modules for processing of different types of input data. For example, the control unit may include a processing utility 600 for processing of the input data where the processing utility 600 includes contrast speckle module 610 and parameter determining module 660, and may also include one or more of frame selector 605, region-spot tracking module 620, defocused speckle monitoring module 630 and rPPG module 640. The control unit generally also include I/O module 520 configured for communicating data and operational commands to the illumination and collection unit and provide network connectivity and/or user interface and storage utility 530 configured for storing intermediate processing data and selected database for determining biological parameters, as well as for storing determined data for further processing when needed.

Generally, the frame selector 605 may be used when the illumination unit 120 is operated by sequential illumination using the laser light source 122 and the field illumination light source 124. More specifically, is such configurations of the system, the collected image data pieces provide multiplexing between frames collected under coherent illumination and suitable for determining and analyzing speckle patterns therein, and frame collected under field illumination and suitable for generating video channel of the subject and his surroundings. The frame selector 605 directs speckle related frames to the contrast speckle module 610 and video channel frames to be processed for tracking as described in more detail below.

The contrast speckle module 610 is configured and operable for receiving input data including at least one sequence of image data piece, and for processing the input data for determining data indicative of one or more biological parameters of the subject. In this connection in image data piece generally include images of an illumination spot S illuminated by coherent illumination and located on inspection region R, preferably directly on skin of the subject. The processing includes determining contrast C of the speckle pattern in each of the image data pieces, where the contrast C is defined as a ratio between the standard deviation σ of pixel intensity and average of pixel intensity I providing:

$$C = \frac{σ}{〈 I 〉}$$

Accordingly, the contrast speckle module 610 generally includes spot tracking module 6010, contrast module 6040 and temporal contrast module 6050, and may also include sub-region segmentor 6020 and alignment module 6030. The spot tracking module 6010 is configured for applying initial processing of a received image data piece for identifying at least one spot region associated with image of the illumination spot. Generally, the illumination spot is relatively monochromatic region having certain speckle pattern therein, associated with light self-interferences.

In some configurations the contrast speckle module 610 may operate for determining contrast of the illumination spot region as a whole, in some other configurations, the contrast speckle module 610 may include sub-region segmentor 6020 configured for segmenting the spot region to a predetermined number of sub-regions and alignment module 6030 configured for aligning relative location of sub-regions between image data piece. Generally, this allows determining contrast of the speckle pattern in each sub-region separately, to provide increased signal to noise ratio and accuracy in determining the selected parameter. It should be noted that as the image data generally corresponds with an image of the illumination spot, different sub-regions of the spot region correspond with different portions of the inspection region R, allowing better processing of biological parameters and identifying blood flow within the inspection region R. The alignment module 6030 is configured for receiving tracking data, typically associated with video channel data, e.g. received from image data pieces collected with field illumination, or image data collected by the second imaging unit 140 and processed by the region-spot tracking module 620. Generally, due to movement of the subject, the system 100 or the surrounding, relative location of the illumination spot S may vary with respect to the inspection region R. The video channel data, e.g. second imaging unit 140 when used or selected frames from the first imaging unit 130, is used for tracking relative location of the subject with respect to the system 100, and of the illumination sport S with respect to the inspection region R. The region-spot tracking module 620 is configured for receiving image data associated with the video channel (e.g. from the second imaging unit 140) and for processing the image data for tracking of these relative locations. If shifts between the illumination spot S and inspection region R are detected, the alignment module may be configured for assigning sub-regions of the spot region in accordance with the detected shift. More specifically, when operating for determining contrast of different sub-region, the present technique may operate for determining sub-region time-contrast functions, and based on the different time-contrast functions, to determine the selected parameters. When the illumination spot S is shifter with respect to the inspection region R between consecutive images/frames, certain sub-regions may still be illuminated allowing re-assignment of the sub-regions and maintaining consistency in following contrast variations for at least one sub-region.

For each sub-region, or for the spot region as a whole, the contrast module 6040 is operable for determining contrast as indicated above. In the case of blood flow, the scattering particles in the blood are constantly moving and therefore decrease the contrast of the speckle pattern in the image. Generally, velocity of the blood is associated on the exact phase of the cardiac cycle, allowing to extract data of the cardiac cycle from contrast variation in image data. As indicated, the contrast module 6040 is operable for receiving data on at least one region or sub-region of the image data, determine average pixel intensity and standard deviation in pixel intensity and accordingly determine contrast of pattern in the region or sub-region and transmit corresponding data to the temporal contrast module 6050.

The temporal contrast module 6050 is configured for aggregating data on varying contrast determined by the contrast module 6040 along time. For each region or sub-region of the image, where the contrast module determines contrast data for each image data piece at the time, the temporal contrast module 6050 collected the contrast data determined for each frame and determined a time-contrast function indicative of variation of contrast in the region or sub-region along monitoring time. As indicated above, such contrast variation is at least partially indicative of blood perfusion and accordingly cardiac activity of the subject. The temporal contrast module 6050 thus determines one or more time-contrast function, generally one for each sub-region of the spot region and transmits data on the time-contrast functions to the parameter determining module 660. The parameter determining module 660 may also receive data from the defocused speckle module 630 and rPPG module 640 for increasing accuracy and generating additional monitoring data that may be used for various machine learning techniques. Generally, the parameter determining module 660 utilizes the one or more time-contrast functions, and possible additional data such as data based on defocused speckle monitoring and/or rPPG data for determining one or more biological and/or biomedical parameters of an individual. Moreover, the parameter determining module 660 may be used for determining existence of a person within dedicated field of view of the system 100.

For example, in some configurations, the technique may utilize monitoring of two or more different inspection regions on an individual, e.g. using illumination unit that produced a plurality of illumination spots. The control unit 500 may be adapted for selecting illumination spots for processing based on image data pieces associated with the video channel and providing input image data indicative of location of the individual. Accordingly, the temporal contrast module 6050 generates two or more time-contrast functions associated with two or more different regions on the individual. The parameter determining module 660 may utilizes such two or more time-contrast functions, in combination with image data indicative of relative location of the two or more inspection regions for determining biomedical parameters such as pulse wave velocity. More specifically, as indicated herein, the time-contrast function provides indication on circulation of blood in an individual. The parameter determining module 660 may compare time difference between circulation measured at the two or more inception regions to determine pulse transmission time and utilizes image data from the video channel for determining pulse wave velocity in accordance with relative distance between the two or more inspection regions. It should be noted that this technique may also be used where one inspection region is monitored using contrast analysis while one other inspection region is monitored using speckle correlation analysis (e.g. using defocused speckle monitoring module 630)

In some additional examples, the parameter determining module 660 may operate for determining blood pressure of the individual in accordance with predetermined relation associated with pulse wave structure. More specifically, the time-contrast function, as measured from selected inspection regions (e.g. neck, face etc.) is indicative of blood pulsation profile of the individual. The parameter determining module 660 may utilize data associated with first- and second-time derivatives of the time-contrast function, quality parameter of the signal indicative circulation pulses and pulse wave velocity parameters, and determining blood pressure of the individual using linear relation between these parameters. Thus, generally parameter determining module 660 utilizes function associated with combination (e.g. linear or non-linear combination) including at least one of first and second time derivatives of the time-contrast function, quality parameter of the signal indicating circulation pulses and pulse wave velocity parameter, with predetermined weight coefficients for determining blood pressure of the individual.

It should be noted that according to some configurations, as indicated above, the illumination unit 120 and its laser light source 122 may be configured to provide a plurality of illumination spots. This may be achieved using a laser array light source or by introducing a diffractive optical elements downstream of the laser light source. In such configurations, the contrast speckle module 610 and its spot tracking module 6010 may operate to determine one or more illumination spot for processing based on image data pieces including a plurality of illumination sports. Further, the contrast speckle module 610 may operate for processing speckle patterns associated with two or more different illumination spots for optimizing signal stability and signal to noise ratio.

It should further be noted that the contrast speckle module 610 may operate on one or more image data pieces (frames) associated with different wavelength of illumination and imaging, thereby providing two or more contrast measures associated with the different wavelengths. In such configurations, the present technique may be used to provide two or more time-contrast functions associated with two or more wavelengths, or a combined time-contrast function associated with summation or averaging (e.g. weighted averaging) of the contrast variations in different wavelength.

As indicated, the present technique may also include defocused speckle monitoring module 630 and rPPG module 640, providing additional monitoring technique over the speckle contrast analysis. In this connection, one of the advantages of using speckle contrast analysis, compared to intensity-based methods such as rPPG, is that the contrast variation is not associated with absorption of light by blood, but rather of scattering properties. This allows the use of illumination in non-visible wavelengths such as infra-red illumination where the absorption coefficient of the blood is relatively low. This is advantageous over visible illumination monitoring by allowing certain penetration through the skin as well as providing relatively eye-safe monitoring. Additionally, contrast of the collected speckle patterns is generally independent of orientation or distance from the inspection region, making the contrast analysis relatively independent of movement of the subject and the surrounding. The contrast of the speckle pattern depends mostly on the velocity of the organ movements rather direction or position thereof. Thus, by collecting image data pieces using short exposure time and utilizing contrast analysis of the collected speckle pattern, the present technique provides effective monitoring of biological parameters while reducing any dependency in mechanical movement or vibrations and allowing operation in noise environment.

Accordingly, the present technique may be highly suitable for monitoring subject within a moving vehicle. For example, system 100 may be located in front of a driver/pilot/operator or any other person/passenger in a moving vehicle and directed for inspection a region of the relevant subject for determining biological parameters such as heart rate, heart rate variability etc., or existence of a person in a selected seat by detecting existence of heart rate. This allows monitoring of health condition of the subject and generating alert if a variation is detected beyond selected threshold. This allows monitoring alertness of drivers as well as medical condition and alerting the driver or vehicle processing unit when condition of the driver requires attention. Further, the technique may be used for scanning of a vehicle interior in response to user instructions to lock the vehicle doors, and determining if any biomedical parameters are identified, suggesting that a person may still be left in the vehicle.

Reference is made to FIG. 3 exemplifying elements of a method for detecting biomedical parameters of an individual according to some embodiments of the present technique. As indicated above, the technique includes directing coherent illumination onto at least one illumination spot 3000. The coherent illumination may preferably be in the form of a plurality of light beams directed toward the inspection region forming a plurality of illumination spots. This arrangement enables the technique to select one or more illumination spots for processing and proceed monitoring the individual's parameters in noisy environment. Using the coherent illumination, data collection is obtained by collecting images 3010 of the inspection region. Generally, separate image may be collected to provide image data (speckle image data) providing speckle pattern under coherent illumination (e.g. at wavelength of 850 nm) and image data visualizing the inspection region (video image data), e.g. the subject and his surrounding, this may be done using first and second imaging unit or in multiplexed image data collection as described using interchangeable field and coherent illumination. The processing may utilize combination of the video image data and speckle image data for detecting one or more faces 3020 within the video image data, or a selected body region for monitoring. Based on the face detection, one or more illumination spots are selected and cropped 3030 from the speckle image data for processing. For improved processing, background of the spot image may be removed 3040 and for each frame, a contrast level is determined 3050 as described above. The contrast level may be improved using dimensional filtering, e.g. by adjusting the speckle pattern into 1 dimension, and/or by additional spot analysis. The collection of speckle contrast measures is collected from a plurality of image data pieces for generating time-speckle function 3060. The required biomedical parameters of the subject may be determined directly from the time-contrast function as indicated above. In some configurations, for improved sensitivity, the present technique may utilize additional data 3070, such as rPPG, defocuses speckles etc. and/or selected post processing for signal enhancement 3080 (shown in dashed line as being used in some embodiments. Using the collected data, the technique may determine heart rate 3092, respiratory rate 3094 and/or inter beat intervals (IBI) 3096. As indicated above, the biomedical parameters may be used for determining presence of a person in selected location (e.g. raising alert when detecting a person in vehicle seat upon locking of the vehicle) as well as for monitoring health and alertness of a person in the vehicle.

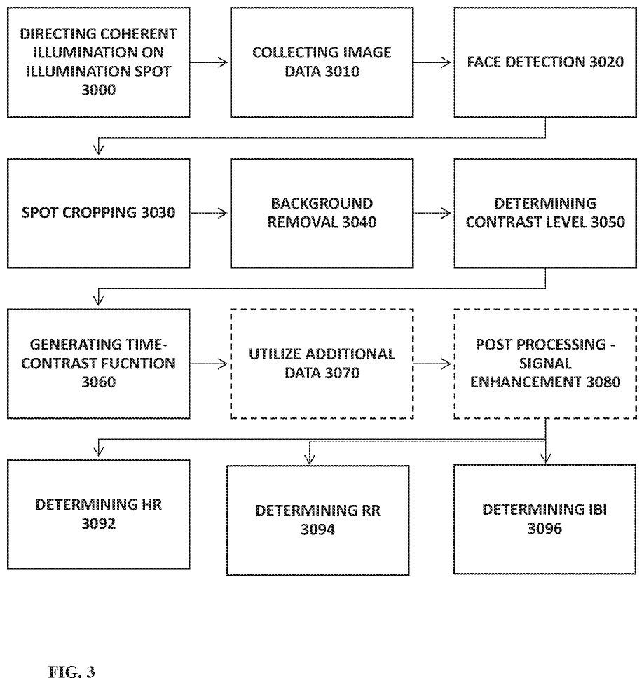

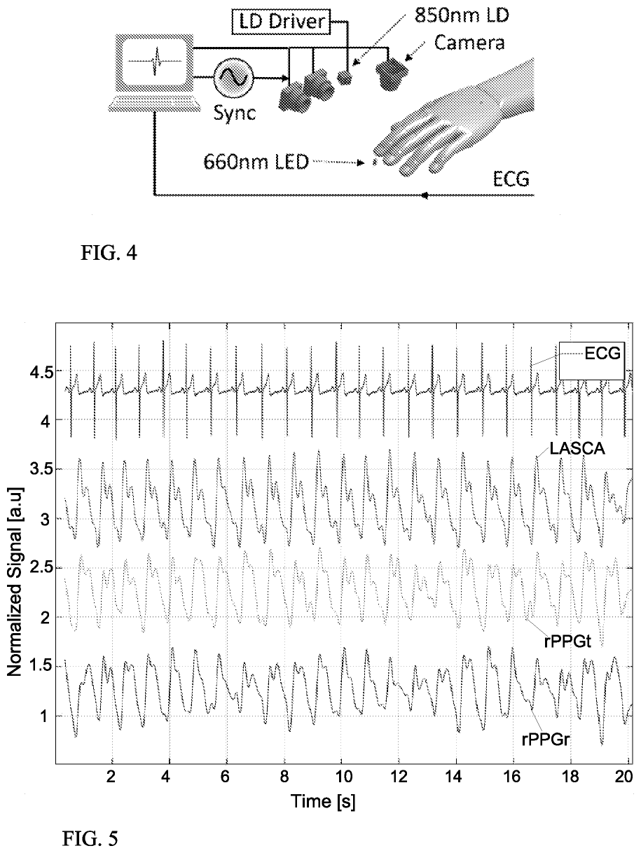

The inventors of the present invention have performed experimental evaluation for determining efficiency of the present technique as compared to existing monitoring techniques. FIG. 4 illustrates the evaluation tests conducted including recording of subject electrocardiogram (ECG) while using remote monitoring including contrast analysis as described above, as well as monitoring the scattered and transmitted light from the inspection region. The ECG was used as a reference device to evaluate the accuracy of the remote methods throughout this experiment. To record the photoplethysmography (PPG) in transmission (rPPGt) a 660 nm light emitting diode was used, located beneath the fingertip, and a camera (BASLER,) equipped with a 12 mm objective (NAVITAR) and a matching filter (FB660-10, Thorlabs). The camera recorded the light transmitted through the finger from which the PPG signal was later extracted by calculating the intensity fluctuations in a subset of the image. This signal is very similar to contact PPG and by itself it has no significant practical value for remote sensing due to the configuration of the collection system. Thus, the PPG signal was recorded to further evaluate the performance of the techniques of the present invention.

To calculate the speckle contrast and remote PPG in reflection (rPPGr) signals, a non-collimated, polarized, 850 nm laser was used to illuminate the entire finger with an intensity of 300 μW/cm². Two additional cameras were used to record the backscattered light from the finger. Both cameras were equipped with a 35 mm objective (NAVITAR) a matching filter (FB850-10, Thorlabs) and a polarizer.

The polarizer is used to reduce noise and is mounted in front of the cameras orthogonally to polarization of the laser light source. Since the light which is scattered from the internal layers of the tissue, where more blood vessels are located, has reduced degree of polarization with respect to the light scattered from the external layers. Accordingly, the use of orthogonal polarization in illumination and collection of light improves quality of detection of light reflected from internal tissue layers providing data of blood perfusion and reducing noise associated with mechanical vibrations.

Generally longer exposure time is beneficial for the intensity-based rPPG monitoring of the blood pulsation. This filters out fast and small movements that alter the intensity of the backscattered light are averaged out. However, the speckle contrast monitoring of the present technique provides improved results in short exposure time, allowing greater variability in contrast between image data pieces and filtering out noise associated with movement. The underlying physics is that the effect of linear movement or tilting of the inspection region contributes to the overall change in the scattering medium on a different time scale compared to the blood pulsation. When recorded with short exposure time, the speckle pattern becomes blurred only by mechanisms that sufficiently change the volume from which the light is scattered, therefore filtering out slow movements

Reference is made to FIG. 5 showing signals recorded by ECG, speckle contrast monitoring (LASCA) as defined herein above, rPPG transmitted light (rPPGt) and rPPG reflected light (rPPGr) techniques. The rPPGt and rPPGr signals were calculated by integrating the intensity of the light passing through (backscattered) from the fingertip. All signals were filtered with a 5th order Butterworth filter with a passband from 0.7 to 10 Hz. To calculate the IBI, the systolic peaks of each signal were identified. The rPPG/LASCA signals were pre-processed, in order to remove IBIs that exceed typical values of 500-2000 ms, resulting from peak misdetection. Generally, the signal recorded by the speckle contrast analysis technique relates to the time-contrast function indicated above.

As shown in FIG. 5, the presently described speckle contrast analysis technique provides heart rate data when the systolic operation can be distinguished allowing monitoring of heart operation as well as blood perfusion. Further, the resulting signal maintains its form showing robustness to movement of the inspection region.

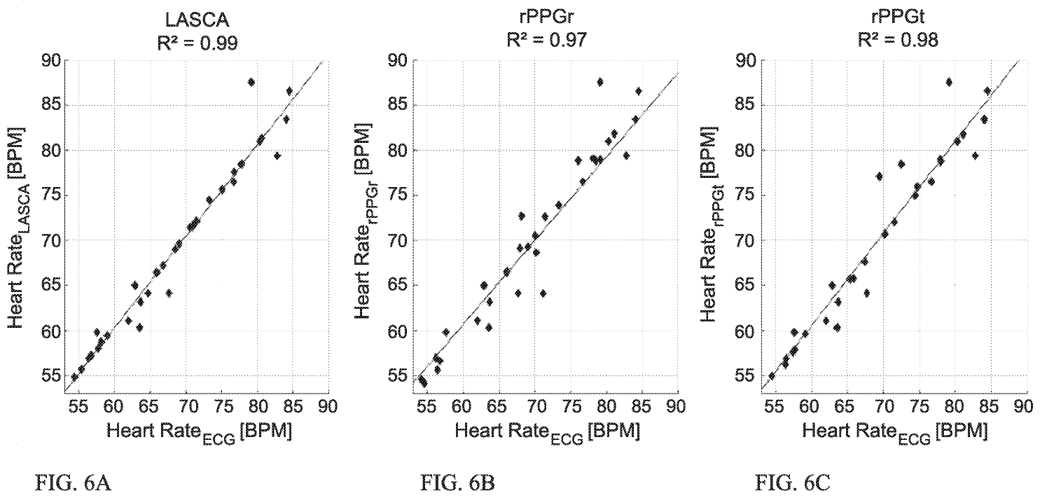

FIGS. 6A to 6C show calculated heart rate determined by the speckle contrast analysis described herein (LASCA), rPPGr and rPPGt techniques compared to ECG over a period of 20 s.

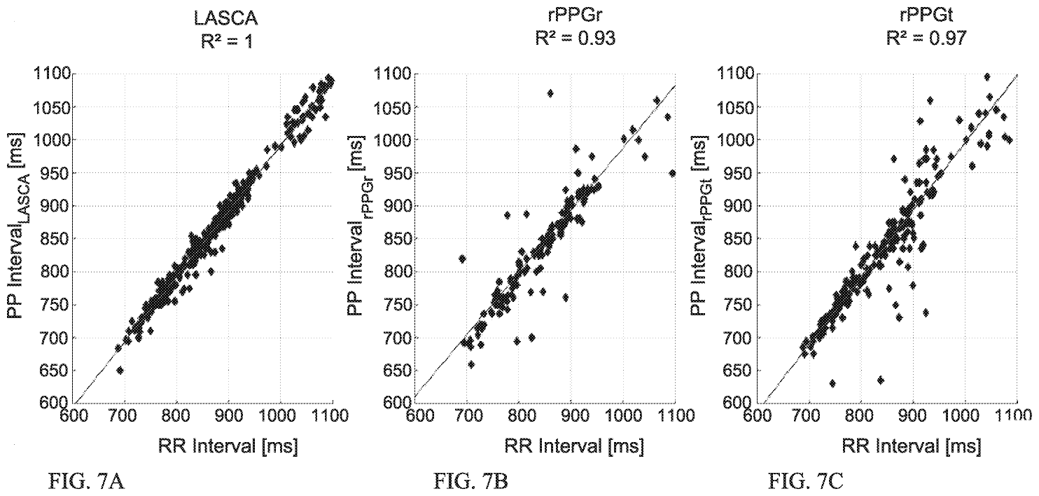

FIGS. 7A to 7C show the correlation inter beat intervals (IBI) as determined by the present technique (LASCA) and by rPPGr and rPPGt with respect to ECG. Here the difference is clear with the Pearson correlation coefficient for the LASCA show perfect correlation while for the rPPGr and rPPGt it is 0.93 and 0.97 respectively

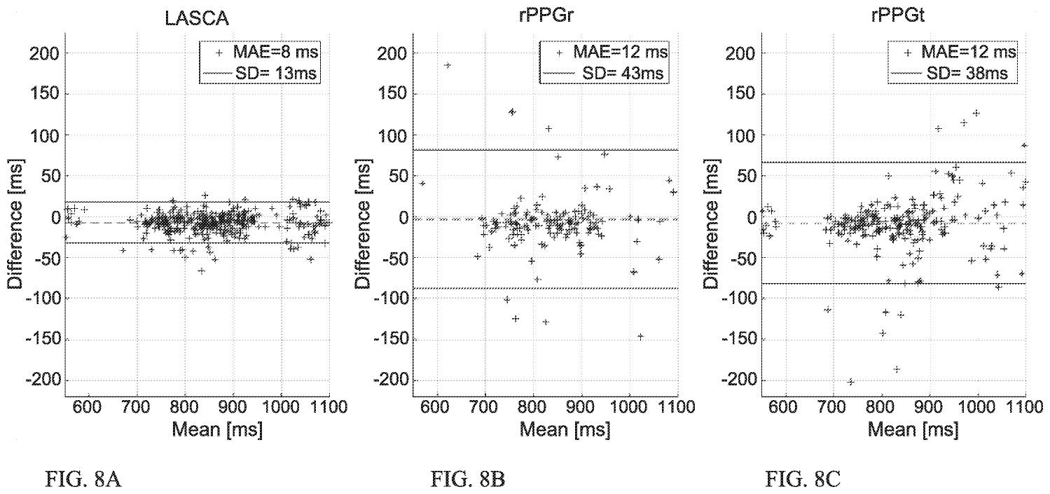

This difference between the methods is seen even more clearly from the Bland-Altman plots presented in FIGS. 8A to 8C. Here the LASCA technique reproduced the results with a median absolute error of 8 ms compared with 12 ms for the rPPGr and RPPGt. Furthermore, the LASCA reproduced the results with a much smaller standard deviation of 13 ms compared to 43 ms and 38 ms for the rPPGr and rPPGt respectively.

As shown, while the optical methods were able to capture the blood pulsation the speckle contrast analysis technique described herein exhibits sharper transitions compared to rPPGr and rPPGt. Furthermore, the shape of the pulses obtained with the present technique is more consistent and is less noisy compared to rPPGt and rPPGr signals. These two properties are extremely important for monitoring IBI and HRV from the signal accurately, and even more important in the case of noisy environment.

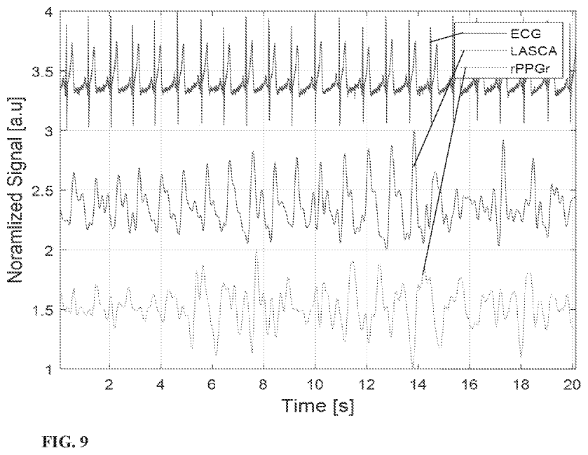

FIG. 9 shows signal collected by speckle contrast analysis (LASCA), rPPGr and rPPGt from a moving finger. In this case the subject was asked to repeatedly move his finger from side to side within field of view of the imaging units. The relevant regions in the image, in both the rPPGr collected image data and the LASCA collected image data, were cropped by simple threshold segmentation of the finger area. The position of pixels related to the finger was analyzed to quantify the displacement, and the movement velocity was measured to be 1.2 mm/s. As shown in FIG. 8, the signal collected by the present speckle contrast analysis technique is relatively robust to the movement noise, while the rPPGr signal was much more corrupted by the movement.

Thus, the present technique provides a system and method for remotely monitoring health parameters of a subject, and specifically heart rate parameters such as heart rate variability and inter beat intervals. The present technique is robust to mechanical noise and movement and provides accurate heart rate data. The results demonstrate that present technique can be used to remotely measure the IBI with excellent accuracy, compared to some established HRV measurement devices. A high correlation was found between the beat to beat intervals derived from the recorded images using the present technique with respect to those derived from ECG. The method is based on common hardware and employs simple algorithms to achieve these results. We also show that LASCA has an advantage over simple intensity-based calculation when the subject moves slowly. Accordingly, the system of the present invention may for example be used for monitoring health parameters in mechanically noisy environments such as moving vehicles or construction areas.

1. A system for monitoring one or more biological parameters of an individual, the system comprising:
an illumination unit comprising at least one coherent light source configured for providing at least one coherent optical illumination beam and for directing said beam onto a selected inspection region on body of said individual;
a light collection unit comprising at least a first imaging unit, said first imaging unit comprising a lens unit and a detector array and configured for collecting light returning from said inspection region to thereby generate at least one sequences of image data pieces associated with said inspection region, at a selected sampling rate; and
a control unit comprising at least one processor, the control unit is configured for receiving input data comprising said at least one sequence of image data piece and for processing the image data pieces for generating data indicative of one or more parameters of the individual, wherein said processing comprises determining contrast variations in image data comprising image data indicative of one or more speckle patterns formed in light returning from illumination spots in said inspection region.

2. The system of claim 1, wherein said processing comprising detecting, within image data pieces of said sequence of image data pieces, region associated with image of illumination spot, said region comprises speckle pattern formed by interference of light returning from the selection illumination spot on the individual's body, and for determining, for at least a portion of said speckle pattern a contrast measure indicative of contrast of speckles in said speckle pattern, thereby generating contrast varying function associated with variation of said contrast measure through time of monitoring, said contrast varying function being indicative of said one or more biological parameters of the individual.

3. The system of claim 1, wherein said first imaging unit is configured for operating at short exposure times, thereby filtering out noise associated with vibrations of said individual.

4. The system of claim 3, wherein said exposure time does not exceed 1000 μs; or does not exceed 500 μs; or does not exceed 100 μs; or does not exceed 50 μs.

5. The system of claim 1, characterized by at least one of the following: said first imaging unit comprises two or more separately operable imaging sub-unit operating with two or more different exposure time profiles; said first imaging unit having a field of view directed at said inspection region, the system further comprises at least a second imaging unit comprising at least one lens unit and detector array, said second imaging unit being configured for collecting illumination from a field of view being larger with respect to said first imaging unit.

6. The system of claim 1, wherein said first imaging unit having a field of view directed at said inspection region, the system further comprises at least a second imaging unit comprising at least one lens unit and detector array, said second imaging unit being configured for collecting illumination from a field of view being larger with respect to said first imaging unit, said control unit comprising a location tracking module configured and operable for receiving image data from said second imaging unit and for tracking location of illumination spot on said inspection region to thereby identify shifts in said illumination spot.

7. (canceled)

8. The system of claim 6, wherein said location tracking module is configured for tagging one or more portions of the inspection region.

9. The system of claim 1, wherein said light source unit comprises one or more light sources providing coherent illumination with two or more different wavelengths within a selected wavelength range, said control unit is configured for receiving and processing image data collected in said two or more different wavelength and for generating two or more corresponding contrast varying function associated with the two or more different wavelengths.

10. The system of claim 1, further comprising at least one additional imaging unit configured for carrying out at least one of the following: collecting defocused image data from the inspection region, and for processing said defocused image data for obtaining additional data indicative of said one or more biological parameters of the individual; and remote photoplethysmographic monitoring for obtaining additional data indicative of said one or more biological parameters of the individual.

11. (canceled)

12. The system of claim 1, wherein said illumination unit further comprising at least one field illumination light source configured for providing wide field illumination of region for inspection; said illumination unit is adapted for operating in sequential pulses of said coherent light source and said field illumination light source.

13. The system of claim 12, wherein said first imaging unit is adapted for collecting image data in synchronization with repeating pulses of said illumination unit thereby collecting image data frames associated with coherent illumination for at least one sequence of image data piece, and image data frames associated with field illumination for generating video channel enabling detection of object or face detection in the field of view.

14. The system of claim 12, wherein exposure time of said first imaging unit is greater than 500 μs, pulse length of said coherent illumination does not exceed 500 μs.

15. The system of claim 1, wherein said at least one coherent light source comprises an array of coherent light sources adapted for providing a pattern of a plurality of illumination spots within a selected field of view.

16. The system of claim 15, wherein said control unit is adapted for selecting one or more illumination spots within said at least one sequence of image data pieces and processing pattern of said one or more illumination spots for determining said one or more parameters.

17. The system of claim 1, wherein said one or more parameters comprise at least one of: heart rate, heart rate variability, respiratory rate, blood pressure and pulse wave velocity.

18. The system of claim 1, wherein said first imaging unit is adapted for wide field imaging, thereby enabling detection of one or more illumination spots from image data collected thereby.

19. The system of claim 1 configured to be mounted within a vehicle for monitoring one or more individuals within the vehicle while moving.

20. The system of claim 19, adapted for determining presence of one or more passengers within the vehicle based on existence of one or more biomedical parameters detected within the vehicle.

21. The system of claim 1 configured for use in wearable device for monitoring one or more parameters of an individual.

22. A method for use in monitoring one or more parameters of an individual, the method comprising: illuminating at least one body portion of the individual with coherent illumination and collecting at least one sequence of image data pieces from said at least one body portion, processing the at least one sequence of image data pieces for determining at least one contrast parameter for each image data piece, thereby determining at least one time-contrast function being indicative of one or more parameters of the individual.

23. The method of claim 22, wherein said collecting at least one sequence of image data pieces comprises operating an imaging unit at high exposure time, not exceeding 1000 μs; or not exceeding 500 μs; or not exceeding 100 μs; or not exceeding 50 μs.

24. The method of claim 22, further comprising monitoring movement of the individual body for following a region associated with illumination spot on at least one body portion of the individual for tracking location of the illumination spot.
